(19) United States
(12) Patent Application Publication
(10) Pub. No.: US 2003/0144578 A1
(43) Pub. Date: Jul. 31, 2003

(54) Anastomosis anchoring device and method

(76) Inventor: J. Kenneth Koster JR., Jacksonville, FL (US)

Correspondence Address: THOMAS C. SAITTA, ROGERS TOWERS BAILEY JONES & GAY, P.A., 1301 RIVERPLACE BLVD., SUITE 1500, JACKSONVILLE, FL, 32207, US

(21) Appl. No.: 10/057,656

(22) Filed: Jan. 25, 2002

(51) Int. Cl. A61B 1/12

(52) U.S. Cl. 600/155

(57) ABSTRACT

The invention is a method and system for anastomosis of a vein graft to an aorta comprising a novel anchoring means for affixing the vein to the aorta wall. The anchoring means for the vein graft comprises an expandible annular wire lattice having short radial projections to secure the lattice in the interior of the vein and longer, flexible prongs which are compressed against the outer wall of the vein while resident within the device, but which expand radially to prevent withdrawal of the vein from the aorta wall after the anchoring means is fully inserted into the aorta. The anchoring means is initially secured to the vein and advanced through a delivery sleeve by use of a balloon catheter.

### BACKGROUND OF THE INVENTION

This invention relates generally to the field of devices and methods used in performing anastomosis, i.e., the joining of a hollow or tubular organ to another hollow or tubular organ, and in particular to anchoring devices and methods used in the surgical joining of a vein graft to the aorta wall. More particularly, the invention relates to self-anchoring anchor means and instruments for accomplishing the anastomosis without suturing.

In coronary bypass surgery, a blocked or damaged segment of an artery is bypassed by attaching a vein graft to the aorta above the blocked point, such that blood flow is routed through the vein graft and around the blockage. In order to attach the graft, a hole must be created in the aorta wall, which may be formed by use of a scalpel but is preferably accomplished using a punch device in order to create a circular opening rather than a slit, as the circular opening is less likely to tear. The punch device typically comprises a sharp cone or bladed disk which creates a small slit in the aorta wall, through which the entire disk is passed. The disk is mounted onto a thin shaft, which is coaxially received by a tubular sleeve member, the end of which is provided with an annular cutting edge or rim. With the aorta wall now disposed between the disk and the sleeve, either the disk is withdrawn into the sleeve or the sleeve is advanced to surround the disk. This shearing operation cuts a circular opening in the aorta wall, and the plug cut from the wall is entrapped within the sleeve and disk when the punch device is removed.

It is necessary to temporarily occlude the opening in the aorta wall in some manner to prevent excessive loss of blood during the anastomosis procedure. In a most basic technique, the surgeon attempts to cover the opening with a finger, but this method is less than optimal. Use of the finger limits the surgeon's dexterity, as it is much easier to attach the vein graft if the surgeon has both hands free and does not need to concentrate on excessive blood loss. Alternatively, a surgical clamp may be applied across the aorta upstream of the opening, which reduces the necessity for the surgeon to rush through the attachment procedure and allows the surgeon free use of both hands, but this technique is problematic in that it stops all blood flow for the period of time necessary to complete the graft, and the pressure from the clamp may damage the aorta or may result in the release of plaque or other debris into the blood stream. Attachment of the vein graft to the aorta wall is usually accomplished by suturing.

It is an object of this invention to provide an anchoring means for securing a vein graft to the aorta, an apparatus for delivering the anchoring means and end of the vein graft into the aorta, and a method of use for same, where the anchoring means, instrument and the methodology provide a simple method for rapid attachment to limit blood loss. It is a further object to provide such an anchoring means, instrument and methodology where secure attachment of the vein graft is accomplished without requiring suturing. It is a further object to provide such an anchoring means, instrument and methodology where the anchoring means is self-securing to the interior wall of the vein graft without suturing, and where the anchoring means is simply and easily attached to the vein graft by use of a balloon catheter. It is a further object to provide such an anchoring means, apparatus and methodology where the anchoring means comprises an expandible annular lattice which is secured to the interior of the vein graft by inflation of a balloon catheter, and where the balloon catheter is also used to advance the end of the vein graft with the anchoring means within a delivery sleeve and through a hole in the aorta wall, and further where the anchoring means is self-expanding once the anchoring means and vein graft end is extended from the delivery sleeve. These objects as well as other objects of the invention not expressly set forth above will be disclosed in the description to follow.

### SUMMARY OF THE INVENTION

The invention is a method and system for anastomosis of a vein graft to an aorta comprising a novel anchoring means for affixing the vein to the aorta wall as well as a novel methodology for insertion of the anchoring means and the end of the vein into the hole in the aorta wall. The anchoring means for the vein graft is self-anchoring and comprises an expandible annular wire lattice having short radial projections to secure the lattice in the interior of the vein and longer, flexible prongs which are restricted and compressed rearward while resident within the delivery sleeve, but which automatically expand radially to secure and prevent withdrawal of the vein from the aorta wall after the anchoring means is fully advanced and extended from the distal end of the delivery sleeve. The anchoring means is secured to the vein prior to insertion into the lateral sleeve by expansion of a balloon catheter, and is advanced through the lateral shaft and cutting sleeve bore by use of the balloon catheter. Alternative embodiments and equivalent elements in addition to those set forth above are also contemplated.

### BRIEF DESCRIPTION OF THE DRAWINGS

FIG. 1 is an illustration of the anchoring means showing the annular lattice ring, the radial projections to secure the anchoring means to the interior of the vein, and the self-expanding anchoring prongs in the non-biased state.

FIG. 2 is an axial view of the anchoring means.

FIG. 3 shows the anchoring means as temporarily retained by the balloon catheter prior to retraction into the vein.

FIG. 4 shows the anchoring means as secured to the vein, and both inserted into the delivery sleeve such that the anchoring prongs are biased to the rear within the delivery sleeve, with the delivery sleeve inserted into the aorta.

FIG. 5 shows the balloon catheter, the end of the vein graft and the anchoring means fully extended from the delivery sleeve, such that the self-expanding anchoring prongs are no longer restricted by the delivery sleeve and are fully expanded in the radial direction.

FIG. 6 shows the anchoring means and vein graft retracted such that the anchoring prongs now contact the interior of the aorta wall, with the lattice ring fully expanded to fill the hole in the aorta wall.

FIG. 7 is a partial view of an alternative embodiment of the anchoring means, where the radial projections are of sufficient length to penetrate through the vein wall and into the aorta wall when the lattice ring is fully expanded, and where the self-expanding anchor prongs are provided with barbed ends which embed in the interior aorta wall.

### DETAILED DESCRIPTION OF THE INVENTION

With reference to the drawings, the invention will now be described in detail with regard for the best mode and the preferred embodiment. In a most general form, the invention is used for the anastomosis of a vein graft to an aorta and comprises an anchoring means which is secured within the end of a vein graft, where the anchoring means is self-anchoring to the aorta wall once extended from a restraining delivery sleeve, such that the vein graft anastomosis is accomplished rapidly and simply without requiring suturing of the vein to the aorta.

A preliminary step in the methodology requires creation of a generally circular hole 92 in the aorta 90 at the attachment site. Techniques and devices known as punches for accomplishing this are well known in the art.

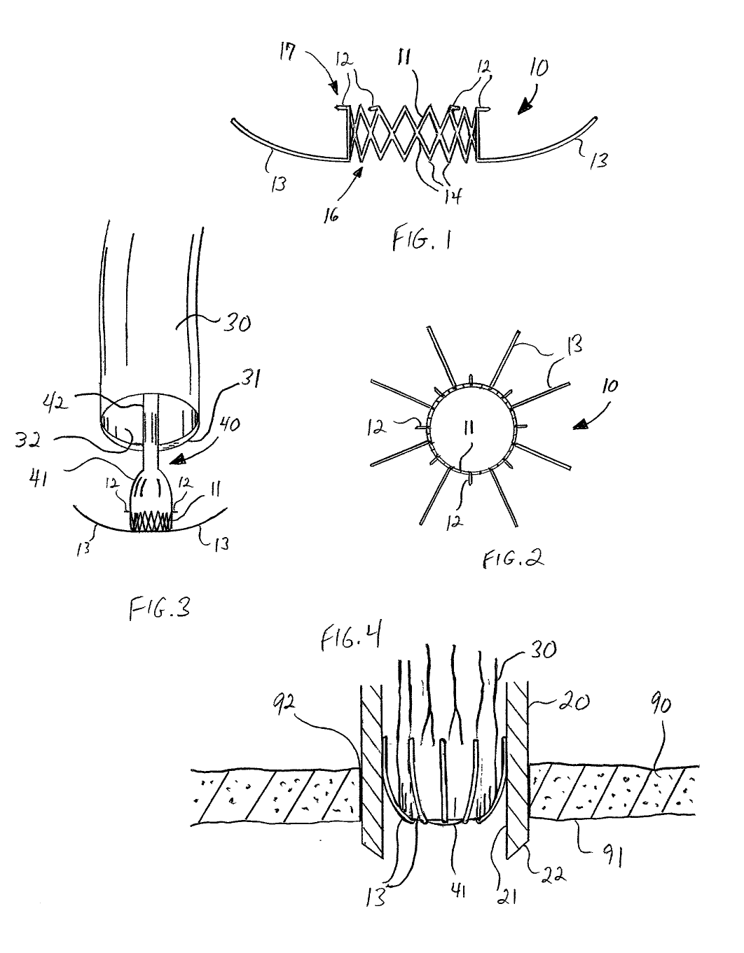

The vein anchoring means 10 is shown in FIGS. 1 and 2, and comprises an annular lattice or lattice ring member 11 formed of meshed, constructed or woven bio-compatible metal, which may be die cut from a metal sheet or formed by joining wire members or the like. The lattice ring 11 is formed with a number of junctions 14 and is sufficiently malleable such that the diameter of the lattice ring 11 can be increased by applying outward force in the radial direction, preferably over the entire circumference, with the height of the lattice ring 11 diminishing as the diameter increases. The material and design of construction is further relatively non-elastic such that the diameter of the lattice ring 11 remains at the increased dimension when the expanding force is removed. The lattice ring 11 is provided with vein securing means comprising a plurality radial projections 12 disposed on its proximal end 17 or on its main body, or both, with the proximal end 17 being the end positioned most interior when the lattice ring 11 is disposed within a vein 30. The radial projections extend outwardly and are used to secure the ring 11 within the vein 30, the radial projections 12 being embedded into interior wall 32 of the vein 30 to prevent relative movement in the axial direction. The lattice ring 11 initially has a compressed configuration with a diameter smaller than the internal diameter of the vein 30. The anchoring means 10 further comprises aorta wall anchoring means comprising self-expanding anchoring prongs 13, which are longer than the radial projections 12 and are connected to the distal end 16 of the lattice ring 11 so as to extend generally radially outward, with the distal end 16 being the end closest to the end 31 of the vein 30 when the lattice ring 11 is disposed within the vein 30. The anchoring prongs 13 may also extend non-radially in a spiraling or tangential direction. The anchoring prongs 13 are sufficiently flexible to allow them to be bent backward on the outside of the vein 92 when the lattice ring 11 is inserted into a tubular delivery sleeve 20, yet retain enough elastic memory such that they will self-expand to resume the extended radial configuration when not so confined. The anchoring prongs 13 are preferably provided with some curvature such that they are generally concave on the side facing the vein graft 30. The anchoring prongs 13 may be circular or polygonal in transverse cross-section.

To secure the lattice ring 11 in the vein 30, the lattice ring 11 is placed onto the deflated head 41 of a balloon catheter 40 with the lattice ring in a reduced diameter configuration. The distal end 16 with the anchoring prongs 13 is positioned toward the distal or free end of the catheter head 41. The balloon catheter head 41 is then expanded sufficiently to retain the lattice ring 11 on the catheter head 41, and the body 42 of the catheter 40 is inserted through the vein 30, as shown in FIG. 3. The catheter head 41 is then pulled into the vein 30 until the anchoring prongs 13 contact the end 31 of the vein 30. The expandible catheter head 41 is then inflated to expand the lattice 11 radially to a point where the projections 12 embed in the interior wall 32 of the vein 30. The vein 30, with embedded anchor means 10 and the catheter head 41, is next inserted into a tubular delivery sleeve 20 in the forward direction, as shown in FIG. 4. The interior diameter of the sleeve bore 21 is chosen so as to be only slightly greater than the diameter of the lattice ring 11 and vein end 31, such that the anchoring prongs 13 are biased rearward into a constricted configuration of greatly reduced overall diameter as the anchoring means 10 and vein 30 are inserted into the delivery sleeve 20.

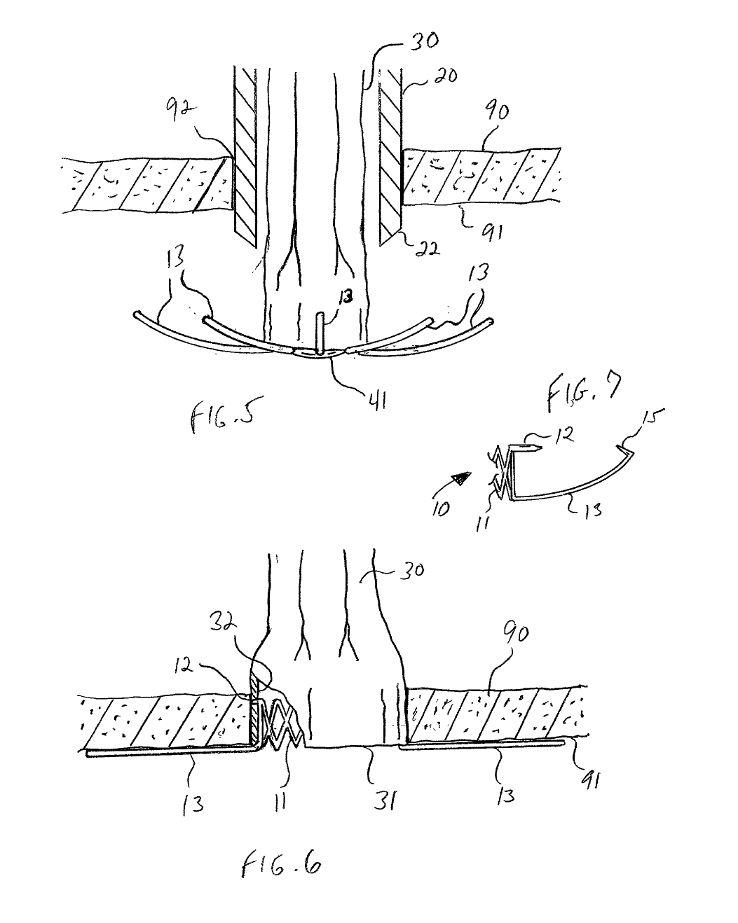

The vein 30 is connected to the aorta 90 as shown in FIGS. 4 through 6. The circular opening 92 in the aorta 90 has been formed by the surgeon. The distal end or tip 22 of the delivery sleeve 20 is inserted into and through the hole 92. The tip 22 of the delivery sleeve 20 is preferably beveled or rounded for ease of insertion. The end 31 of the vein 30 is now advanced by pushing the catheter body 42 forward through the delivery sleeve 20, as shown in FIG. 5. The catheter head 41, anchoring means 10 and vein 30 are advanced a sufficient distance so as to be positioned internally within the aorta 90 and extending freely and completely beyond the sleeve tip 22. When the free ends of the anchoring prongs 13 clear the delivery sleeve tip 22, they automatically spring outward to extend radially within the aorta 90, as shown in FIG. 5. The catheter head 41 is next retracted slightly so that the anchoring prongs 13 abut the interior wall 91 of the aorta 90, and is then fully inflated to insure that the lattice ring 11 is securely embedded and maximumly expanded to present the largest possible flow opening through the vein 30 and to insure that the exterior of the vein 30 abuts and is lodged in the hole 92 such that no opening remains external to the vein 30 for loss of blood. The catheter head 41 is deflated such that it no longer grips the lattice ring 11 and is withdrawn from the vein 30 and from the delivery sleeve 20, which is also withdrawn. The entire vein 30 now passes through the delivery sleeve 20 and remains attached to the aorta 90.

An alternative embodiment of the anchoring means 10 is shown in FIG. 7, such that the radial projections 12 may be sized to extend completely through the wall of the vein 92 and into the aorta 90 itself when the lattice ring 11 is fully expanded. Also, the anchoring prongs 13 may be curved or angled at the free ends to define barbed ends 15 or like projections which physically embed into the aorta interior wall 91 when the anchoring means 10 is retracted to abut the interior wall 91.

It is contemplated that certain equivalents and substitutions for elements set forth above may be obvious to those skilled in the art, and therefore the scope and definition of the invention is to be as set forth in the following claims. Likewise, the embodiments of the invention as set forth above are not expressed for the purpose of limiting the invention, and alternative or similar embodiments of an equivalent nature falling within the scope of the claims are contemplated.

I claim:

1. An anastomosis anchoring device for securing the end of a vein graft to an aorta, said anchoring device comprising:
a radially expandible lattice ring member;
radial projections extending from said lattice ring member, said radial projections adapted to embed in a vein graft; and
self-expanding anchoring prongs extending from said lattice ring member, said anchoring prongs adapted to abut the interior surface of an aorta, said anchoring prongs being of greater length than said radial projections.

2. The device of claim 1, wherein said lattice ring member comprises a distal end and a proximal end, and wherein said anchoring prongs extend from said distal end of said lattice ring member.

3. The device of claim 2, wherein said radial projections extend from said proximal end of said lattice ring member.

4. The device of claim 1, wherein said lattice ring member is formed of a material which is radially expandible to a configuration of increased diameter such that said lattice ring retains such increased diameter when any radially expanding force is removed.

5. The device of claim 1, wherein said anchoring prongs are formed of a material whereby said anchoring prongs may be biased from the expanded configuration toward said lattice ring and further whereby said anchoring prongs automatically resume such expanded configuration when any biasing force is removed.

6. The device of claim 1, further comprising a removable delivery sleeve comprising a bore and a tip, wherein said delivery sleeve is adapted to temporarily receive said lattice ring member disposed within said bore such that said anchoring prongs are biased toward said lattice ring member.

7. The device of claim 1, wherein said radial projections are sized to extend completely through said vein graft.

8. The device of claim 1, said anchoring prongs further comprising barbed ends.

9. An anastomosis anchoring device for securing the end of a vein graft to an aorta, said anchoring device comprising:
a radially expandible lattice ring member comprising a distal end and a proximal end, said lattice ring member formed of a material which is radially expandible by a radially expanding force to a configuration of increased diameter such that said lattice ring retains such increased diameter when such radially expanding force is removed;
vein securing means for securing said lattice ring member to said end of said vein graft, said vein securing means comprising radial projections extending from said lattice ring member, said radial projections adapted to embed in said vein graft; and
aorta wall anchoring means to secure said lattice ring to said aorta wall, said aorta wall anchoring means comprising self-expanding anchoring prongs extending from said lattice ring member, said anchoring prongs adapted to abut the interior surface of an aorta, said anchoring prongs being of greater length than said radial projections, said anchoring prongs formed of a material whereby said anchoring prongs may be biased by a biasing force from the expanded configuration toward said lattice ring and into a constricted configuration, and further whereby said anchoring prongs automatically resume such expanded configuration when such biasing force is removed.

10. The device of claim 9, wherein said anchoring prongs extend from said distal end of said lattice ring member.

11. The device of claim 10, wherein said radial projections extend from said proximal end of said lattice ring member.

12. The device of claim 9, further comprising a removable delivery sleeve comprising a bore and a tip, wherein said delivery sleeve is adapted to temporarily receive said lattice ring member disposed within said bore such that said anchoring prongs are biased toward said lattice ring member.

13. The device of claim 9, wherein said radial projections are sized to extend completely through said vein graft.

14. The device of claim 9, said anchoring prongs further comprising barbed ends.

15. An anastomosis method of attaching a vein graft to an aorta wall comprising the steps of:
creating a generally circular hole in said aorta wall;
inserting an anchoring device into the end of said vein graft, said anchoring device comprising a radially expandible lattice ring member, vein securing means to secure said lattice ring member to said vein graft, and aorta wall anchoring means to secure said lattice ring to said aorta wall, whereby said vein securing means and said lattice ring member are disposed internally to said vein graft and said aorta wall anchoring means are disposed externally to said vein graft;
expanding said lattice ring member to embed said vein securing means in said vein graft;
inserting said anchoring device and said vein graft into a delivery sleeve to constrict said aorta wall anchoring means;
inserting said delivery sleeve into said hole in said aorta wall;
advancing said vein graft end and said anchoring device from said delivery sleeve such that said aorta wall anchoring means are no longer constricted;
withdrawing said delivery sleeve from said hole and retracting said vein graft such that said aorta wall anchoring means abut said aorta wall internally and said lattice ring member is disposed within said hole; and
expanding said lattice ring such that said vein graft end abuts said hole.

16. The method of claim 15, further comprising the steps of providing a balloon catheter with an inflatable head and positioning said catheter head within said lattice ring member;
wherein said step of inserting said anchoring device into said end of said vein graft comprises the steps of inflating said catheter head to retain said lattice ring member thereon and inserting said balloon catheter through said vein graft to properly locate said lattice ring member within said vein graft end;
wherein said step of expanding said lattice ring member to embed said vein securing means in said vein graft comprises the step of further inflating said catheter head;
wherein said step of advancing said vein graft end and said anchoring device from said delivery sleeve comprises the step of advancing said catheter head from said delivery sleeve;
wherein said step of retracting said vein graft comprises retracting said catheter head;
wherein said step of expanding said lattice ring such that said vein graft end abuts said hole comprises further inflating said catheter head; and
further comprising the step of deflating said catheter head and withdrawing said catheter head through said vein graft.
